(19) United States
(12) Patent Application Publication
(10) Pub. No.: US 2025/0260896 A1
(43) Pub. Date: Aug. 14, 2025

(54) MEDICAL VISUALIZATION SYSTEM, MEDICAL OPERATING SYSTEM AND METHOD FOR VISUALIZING A VIDEO IMAGE DATA STREAM

(71) Applicant: Schölly Fiberoptic GmbH, Denzlingen (DE)

(72) Inventors: Daniel HÄRTER, Emmendingen (DE); Stefan SCHRÖER, Freiburg (DE); Alexander KÖHLER, Freiburg (DE)

(73) Assignee: Schölly Fiberoptic GmbH, Denzlingen (DE)

(21) Appl. No.: 19/041,151

(22) Filed: Jan. 30, 2025

(30) Foreign Application Priority Data
Feb. 8, 2024 (DE) 102024103538.2

(51) Int. Cl. H04N 23/661

(52) U.S. Cl. CPC H04N 23/661

(57) ABSTRACT

A system architecture for a medical visualization system (1) is provided, having a central control module (CCM) (4), using which numerous components (3, 2, 6 and possibly 23), in particular a camera control unit (3) and optionally peripheral devices (6), can be accessed in a controlling manner, in order to be able to exert a central control function in the sense of a “surgical cockpit” in this manner. With the aid of the CCM (4), the user, for example, with the aid of a touchscreen (15), can control and regulate in a controlling manner all important functions of the visualization system (1), wherein the visualization system (1) can also be modularly expanded using further components, which can then each be newly connected to the CCM (4).

### CROSS-REFERENCE TO RELATED APPLICATIONS

This application claims priority from German Patent Application No. 10 2024 103 538.2, filed Feb. 8, 2024, which is incorporated herein by reference as if fully set forth.

### TECHNICAL FIELD

The invention relates to a medical visualization system, which comprises at least one camera control unit and a central control module. The camera control unit is configured here to read out image signals or image data from at least one associated/assigned image recording device (for example, one or more video cameras) and to generate respective processed image data from the respective (received) image signals/image data. A user can access the at least one camera control unit in a controlling manner via the central control module, in order to have the respective (processed) image data generated by the respective camera control unit displayed on at least one monitor. In other words, the central control module then instructs the respective camera control unit via a control connection (this can be implemented wirelessly or by means of a cable) to output the respective processed image data on an assigned monitor. However, the central control module does not have to receive the actual image data for this purpose, as will be explained in more detail hereinafter, i.e. the central control module itself does not transmit any image data. In addition, this system can also comprise at least one such associated image recording device, for example, in the form of an endoscope, an exoscope, or a microscope, and/or an associated monitor.

Furthermore, the invention relates to an associated medical operating system which, in addition to such a visualization system, comprises at least one peripheral device (such as a light source and/or an anesthesia device and/or an insufflator and/or a pump and/or an OP instrument).

Finally, the invention also relates to a method for visualizing at least one video image data stream, which is recorded using at least one image recording device (for example, an endoscope/exoscope/microscope as described above), wherein this image recording device can be part of a visualization system according to the invention. In this method, processed image data can be generated with the aid of at least one camera control unit in the context of preprocessing from image signals or image data of the at least one image recording device. These processed image data, or advanced image data generated therefrom, can then be displayed on a monitor in the form of the video image data stream.

### BACKGROUND

Such visualization systems as described at the outset are already in use in surgical interventions, for example, on the basis of a video endoscope as the image recording device. In general, such a system is equipped with a higher-order controller, which is often designed in the form of a separate camera control module (often designated as “camera control unit”=CCU), so that a user can influence the imaging and therefore ultimately the visualization of the recorded image data according to their wishes with the aid of this camera control module.

EP 2 749 201 B1 and EP 3 335 619 B1 each describe a modular endoscopic video system having one or more cameras, which are read out by respective “input modules” in order to generate image data. The video system also comprises a higher-order control module, which can access the respective “input modules” in a controlling manner. This higher-order control module then receives image data, further processes these received image data, and finally transfers these data to a monitor for display. The disadvantage of this system is above all the complex bidirectional communication between the control module and the respective “input module”, since these two communication partners initially have to come to an agreement on a common standard for transmitting the image data beforehand. In particular, so-called “module identifiers” have to be transmitted to the “control module” for this purpose.

### SUMMARY

Proceeding therefrom, it is the object of the invention to propose a system architecture which enables easy adaptability of the visualization system to customer wishes and at the same time overcomes previously known disadvantages of prior systems. In particular, it is to be possible by means of the system architecture to still be able to use existing components as part of the visualization system but at the same time be able to expand the visualization system modularly with new parts or peripheral devices at any time, if this is required in the application.

One or more of the features disclosed herein are provided to achieve this object.

In particular, a visualization system as described at the outset is proposed to achieve the object mentioned at the outset, which is distinguished in that the at least one camera control unit can be instructed to transmit the image data to a monitor in the context of a central control function with the aid of the central control module via a respective control connection. In other words, the central control module is thus configured to instruct the respective camera control unit via the respective control connection so that it transmits image data, which the respective camera control unit has received from the image recording device assigned thereto and subsequently processed, to a monitor.

As will be explained in more detail later, in the case of this transmission, a further module can receive the already processed image data from the camera control unit and further process these data once again (for example, to form “advanced image data”), before these image data which have been processed once again are ultimately output in the form of a video image data stream on the monitor.

In contrast to previously known approaches, for example, as described in EP 2 749 201 B1 and EP 3 335 6119 B1, in the achievement of the object according to the invention, the respective video image data stream is not output by a higher-order control module, but rather by the respective camera control unit (CCU) itself to the respective display/the respective monitor for visualization, wherein this can take place mediated via a further (noncentral) module, such as an advanced video image processing module (AVM). The central control module according to the invention nonetheless exerts the central control within the visualization system and thus acts as the master of the system, which instructs other components as slaves. The central control module according to the invention does not output any image data to a monitor, however.

The central control module and the respective camera control unit are preferably each designed here as separate structural units. This is because in this way the overall system can be adapted modularly very easily to customer wishes, in particular scaled.

A visualization system according to the invention is accordingly distinguished by a specific system architecture, which is in turn distinguished by the fact that a processor-based central control module (also designated here as a “central control module=CCM”) is provided as a separate structural unit, which exerts the central control and acts in a controlling manner on at least one camera control unit (CCU).

The respective CCU can be designed as a structural unit separate from the CCM, for example, if a CCU is already provided. The respective CCU reads out image signals or image data already supplied by the camera from at least one associated camera and further processes these data accordingly to form processed image data. The at least one image recording device mentioned at the outset as part of the visualization system can therefore implement a camera, in particular a video camera.

A visualization system according to the invention can offer, for example, network-based functions such as DICOM (“digital imaging and communications in medicine”) and/or a central control function and/or, for example, functions such as the automatic creation of worklists (“automated worklists”) to the user. With the aid of a CCM according to the invention, the utility of the overall system can therefore be significantly increased for the user. This is valuable in particular for medical applications, because the requirements for the documentation are becoming higher and higher there, so that there is a need to archive video image data, which were recorded using the visualization system, in the form of so-called DICOM database objects. DICOM standardizes here both the format for storing the data and the communication protocol for their exchange, for example, within a computer network of a hospital. The DICOM standard is already used in products in imaging or image processing systems such as digital x-ray, MRI, CT, or sonography. DICOM additionally often forms the foundation for digital image archiving in hospitals with the aid of the so-called PACS (“picture archiving and communication system”).

It can therefore be provided that the central control module is designed so that it can implement an access to an external server at least indirectly (thus in particular mediated via a further component of the visualization system), in order to archive DICOM objects and therefore recorded video image data on the server. In this case, however, the central control module itself does not necessarily have to receive or process the image data, but rather the central control module (CCM) can instruct, for example, a CCU of the system or another system component subordinate to the central control module to generate and transmit DICOM objects to an external instance (or can be configured for this purpose).

In such a design, the communication between the CCM and the respective CCU can therefore be made bidirectional. In such a case, however, image data are not exchanged between the CCM and the respective CCU, but rather network data, while image and/or video files can be transmitted via the network to the CCM, in particular via a separate transmission path (in which the CCU does not participate). In this approach, in particular real-time data thus do not have to be exchanged between the CCM and the respective CCU, as in previously known approaches, which greatly simplifies the system architecture. In contrast to previously known approaches, the bidirectional communication between CCM and CCU is therefore restricted to network data, but does not extend to real-time-capable video or image data.

A visualization system according to the invention can also comprise a module for advanced video processing, in particular in the form of an “advanced video processing module” (AVM). This AVM can receive already preprocessed image data from the respective CCU and supply these data to a downstream video processing function or implement such a downstream video processing function. Advanced image data can be generated by the downstream video processing function carried out by the AVM. These advanced image data can then be output by the AVM at a corresponding display unit/a monitor, in order to thus enable advanced visualization.

As will be explained hereinafter, “advanced image data” can be understood in particular as image data which are supposed to be/have to be visualized by means of the following means: geometric scaling of video images and user-specific spatial arrangement/visualization of the image data, for example, as “side-by-side”, PiP (“picture-in-picture”), PoP (picture out of picture), or by means of “window throwing” (this is understood as manual shifting back and forth of the respective window as in typical office software applications). Such “advanced visualization” can also be understood as offsetting different video signals or video channels, for example, to achieve a specific desired representation (for example, in “hyperspectral imaging” applications).

Overlay technologies are also important: for example, an IR signal in the case of fluorescence imaging as an overlay on a video image.

Such a downstream video processing function can accordingly comprise, for example, scaling and/or a spatial arrangement of at least two video image data streams which are supplied by different CCUs of the system, or, for example, a (nearly arbitrary) offsetting of such different video image data streams. The video image data streams can preferably be transmitted here by means of video signals from the respective CCU to the AVM.

Using the AVM, in particular functions of “advanced imaging”, for example, overlaying multiple live video images to form an artificial overall image and/or augmenting video images (“augmented images”) with additional objects and/or with additional information can be implemented.

Therefore, it can be characteristic for a visualization system according to the invention, among other things, that the CCM has a controlling effect on a respective one of at least two different CCUs via each of at least two different control paths. Of course, a CCM according to the invention can also already be reasonably used with only one CCU. Due to the proposed type of actuation, the CCM can trigger and/or control a specific signal processing function in the respective CCU in each case, for example, to generate a respective image data stream (preferably in the form of a video image data stream). Such a (video) image data stream (“processed image data i”) can then be supplied to the AVM, wherein the CCM (for example, via a path “iv”—cf. FIG. 3 in this regard) can also have a controlling effect on the AVM here.

Each of the CCUs used in the system can have a separate processor here, configured for processing image signals of the respective associated image recording device/camera.

And the AVM can also have a separate processor and associated software, wherein the processor of the AVM is configured to process the different video image data streams (transmitted/incoming from the respective CCU) and to generate at least one advanced image data stream therefrom, preferably in the form of a live video image data stream.

The CCM can also have a separate input unit and a separate display device (ideally even combined in the form of a touchscreen). In this way, the CCM can visualize many types of information, which are required, for example, to perform desired settings, to the user and moreover the user can input control commands via the input unit/the touchscreen into the CCM, so that it generates corresponding control commands therefrom. In other words, the user can therefore, via the CCM, exert/execute a central control function within the visualization system and at the same time access (possibly all) components of the system in a controlling manner.

The medical visualization system described above can also be refined as follows, as can be seen on the basis of the dependent claims:

For example, the central control module can be configured to access the at least one camera control unit and at least one peripheral device in a controlling and regulating manner via a local network. For this purpose, in particular multiple respective bidirectional control connections between the central control module and further devices of the visualization system can be formed in the network.

It is furthermore preferred if the central control module is not configured to receive image data, since this substantially simplifies the communication in the system. Rather, the central control module can be configured only to regulate a flow of image data (in particular in the form of a video image data stream) between individual components of the visualization system in a controlling manner. This control can in particular comprise a specification of the rate at which the respective video image data stream is to be output and/or in which video image data format.

It is furthermore preferred if the central control module is configured to request parameters from the at least one camera control unit and/or from the at least one peripheral device via respective direct control connections, i.e. in particular without a detour via another device being taken in this request.

The central control module can furthermore be configured, in the context of a gateway function, to provide an Internet access and/or an access to a local database, in particular to a server, and/or an access to a cloud service (thus a server which is only accessible via the Internet) for the at least one camera control unit and/or for at least one peripheral device. In this way, the central control module can enable a communication between such external instances (Internet/cloud/database) and the respective device/camera control unit, and in particular without the respective device having to communicate bidirectionally with the external instance (for example, the database or the cloud) for this purpose. It can also be provided here that, for example, the respective camera control unit or one of the peripheral devices can itself send data, in particular image data, to the external instance via an additional data connection (cf. in this regard FIG. 3, path i)), wherein this sending preferably also takes place in reaction to an instruction by the central control module.

The central control module can also be configured to offer network-based functions such as DICOM (“digital imaging and communications in medicine”) and/or a central control function for the visualization system and/or at least one auxiliary function, for example, an automatic creation of worklists, to a user of the visualization system. In such an implementation, it is then preferred if the user can operate this function/these functions via a user interface (which can in particular be an input unit, such as a touchscreen) of the central control module.

The central control module can therefore have a separate input unit and/or a separate display device in order to enable the central control function described at the outset. In particular, for example, a user can access the at least one camera control unit (thus in particular multiple differently designed camera control units each having adequate control commands) in a controlling manner via the input unit.

It can furthermore be provided that at least one piece of external information, which is acquired from the camera control unit and/or from a peripheral device connected to the control module, is displayable on the display device. Such pieces of information can be, for example: an operating state or an operating parameter of a peripheral device connected to the control module; vital data of a patient; a measured pressure at an insufflator (connected to the CCM as a peripheral device) or a pressure of a pump (such pieces of information can be critical, since there is a risk for the patient from a specific pressure); or, for example, current settings of an HF surgery instrument which is currently being used to cut tissue. Such a design substantially improves the usage properties of the overall system, because the user can thus have important pieces of information from the periphery of the visualization system displayed as needed on the display device of the control module.

In addition, the CCM and the display device can be designed so that peripheral devices are actuatable via the CCM, for example, a pump and/or an insufflator, in particular to be able to specify a specific pressure. The same applies for the actuation of an HF surgery instrument, since HF operating parameters can thus be easily specified.

A visualization system according to the invention can also comprise a module which is configured for advanced video processing (“advanced video processing module”=AVM). This module/the AVM can thus be configured to receive already preprocessed image data from the at least one camera control unit and generate advanced image data therefrom by means of a downstream video processing function. The advanced video processing module can also output the advanced image data directly to a connected monitor in this case.

It is particularly preferred in this case if the advanced video processing module (AVM) is designed as a structural unit separate from the central control module.

Furthermore, the central control module (CCM) can access the advanced video processing module (AVM) in a controlling manner via a control connection, in order to also be able to exert a control function there. The transmission of the image data from the camera control unit to the (respective) monitor can thus in particular take place mediated via the AVM, and preferably without the respective image data being transmitted here via the central control module/to the central control module.

The described advanced video processing module (AVM) can also be configured to receive multiple video image data streams, in particular in the form of video signals, from assigned camera control units of the visualization system (thus from at least two such camera control units) and to offset and/or scale these received video image data streams with one another and/or specify a spatial arrangement of at least two of these received video image data streams.

The AVM can also be configured to overlay multiple live video images and/or augment at least one live video image data stream with additional objects and/or additional information.

As mentioned at the outset, a medical operating system having one or more of the features disclosed herein is also provided to achieve the object. It is provided in this case that the at least one peripheral device of this operating system (which can be designed as described at the outset) is readable and/or controllable, thus in particular operable by a human, via the central control module. Such reading/controlling/operating can in particular be implemented with the aid of an input unit and/or a display device of the central control module. Very particularly advantageously, this is achieved, for example, using a touchscreen, using which both inputs can be made and data/parameters can be displayed.

It is furthermore also to be noted that a medical operating system can of course comprise a medical visualization system as described above and/or as claimed in one of the claims directed to such a visualization system.

Furthermore, a method for visualizing at least one video image data stream having one or more of the features disclosed herein is also provided to achieve the object. In particular, in a method of the type mentioned at the outset, it is therefore proposed that in the context of a central control function, with the aid of a central control module (which can be designed as described above), the at least one camera control unit (this can be one or more devices) is instructed via a respective control connection to transmit the processed image data to the monitor. “Instruct” can in particular be understood here to mean that the forwarding of video image data by the camera control unit takes place in reaction to and in accordance with a control signal, which the central control module sends to the respective camera control unit.

This method can also be refined as follows:

For example, the mentioned advanced image data can be generated with the aid of an advanced video processing module (AVM-this can be designed as described above) in the context of a downstream video processing function. Subsequently, the advanced image data thus generated can be transmitted from this advanced video processing module (AVM) to the monitor, on which the video image data stream is to be displayed. This transmission and/or the downstream video processing function can in particular be controlled and/or adapted (in particular according to desires/input commands of a user) here via a control connection with the aid of the central control module. In other words, the control of the manner of the described downstream video processing function can thus be centrally controlled with the aid of the advanced video processing module by a user via the central control module and can be adapted according to their current wishes, for example.

In the described visualization of the image data or the advanced image data, access can be made in a controlling manner to the at least one camera control unit and/or to the advanced video processing module via the/mediated by the central control module, and preferably in consideration of a piece of external information which is supplied by a peripheral device. This external information can be displayed to a user, for example, on a display device of the central control module. Such external pieces of information can be required, for example, when so-called “case data” are to be processed on the CCM, thus, for example, when “worklists” are acquired from an external instance (for example, from KIS (online)). There are also possible offline scenarios: For example, the manual creation of a worklist and/or the manual application of “case data” of a patient. All such operations, in particular saving and/or archiving “case data” and also deleting data on a CCU can be effectuated using the CCM if it is configured for this purpose.

In the method, it can furthermore be provided that access is made in a controlling manner via the central control module to a peripheral device connected (via cable or by means of a radio connection) to the central control module. In this way, in particular imaging with the aid of the at least one image recording device can be influenced, for example, if the peripheral device provides an illumination light or an excitation light for the imaging.

In other words, the central control module (CCM) can thus be used as a “surgical cockpit” and can display important parameters of a peripheral device to the user. However, the user can also control, for example, a light source, which is used in the imaging using the respective image recording device of the visualization system, via the central control module.

### BRIEF DESCRIPTION OF THE DRAWINGS

The invention will now be described in more detail on the basis of exemplary embodiments, but is not restricted to these exemplary embodiments. Further designs of the invention can be derived from the following description of a preferred exemplary embodiment in conjunction with the general description, the claims, and the drawings.

In the following description of various preferred embodiments of the invention, elements corresponding in their function receive corresponding reference numerals even with different design or shaping.

In the figures:

FIG. 1 shows a visualization system as is already used in the prior art,

FIG. 2 shows a first visualization system designed according to the invention,

FIG. 3 shows a second visualization system, likewise designed according to the invention, and

FIG. 4 shows a typical application situation, in which a visualization system according to the invention, in particular illustrated as in FIG. 2 or FIG. 3, can be used as part of an operating system.

### DETAILED DESCRIPTION

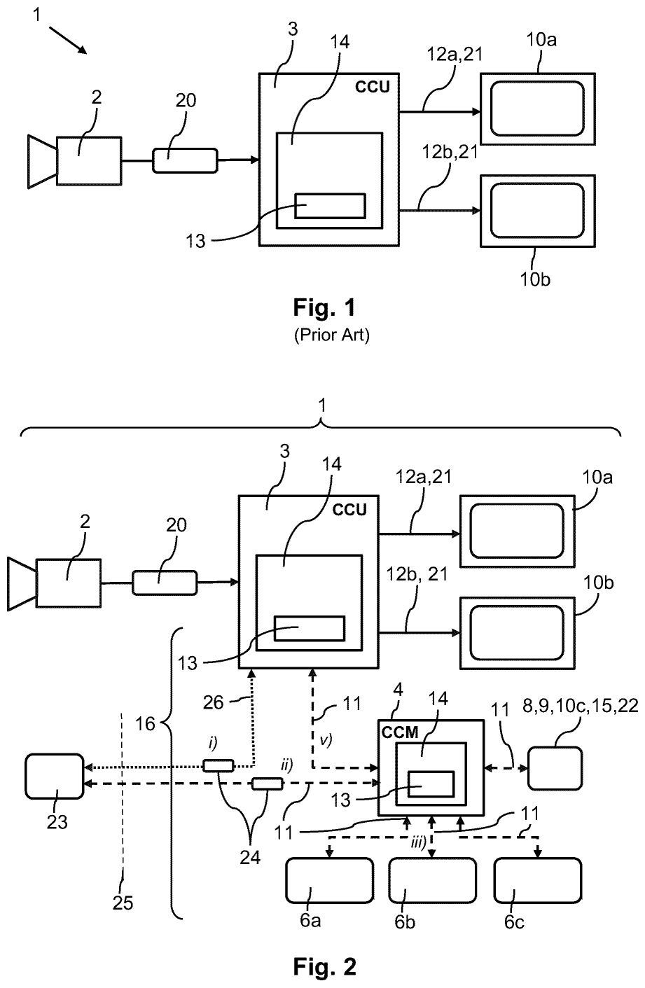

FIG. 1 shows a previously known visualization system 1 from the prior art, which comprises an image recording device 2 in the form of a video camera, which generates (unprocessed) image data 20 and transmits them to a camera control unit 3 (CCU). The CCU 3 has a separate processor 14 and associated software 13, in order to further process the incoming image data stream 20 from the image recording device 2. The CCU 3 can in this way output two different image data streams of in each case processed image data 12a, 12b in the form of respective video signals 21 at an assigned respective monitor 10a, 10b, so that these two video image data streams can be displayed on the respective monitor 10a, 10b.

FIG. 2 shows a system architecture of a first medical visualization system 1 according to the invention. The above-described structure having an image recording device 2 can also be seen here, which transmits a stream of not yet processed image data 20 to a CCU 3 for further image processing. The CCU 3 outputs two different image data streams 12a, 12b each made up of image data processed independently of one another at the respective monitor 10a, 10b. However, the system 1 also additionally comprises a central control module 4, which is connected via numerous control connections 11 not only to the single camera control unit 3, but also to multiple peripheral devices 6a, 6b, 6c, to a display device 9 in the form of a touchscreen 15, and also via the path ii) to an external server 23.

In the context of a central control function, the central control module 4 instructs the CCU 3 via the path v) to process the received unprocessed image data 20 and transmit it in the form of processed image data 12a, 12b to the respective monitor 10a, 10b. As can be seen well on the basis of FIG. 2, however, the processed image data 12 do not arrive here at the central control module (CCM) 4, since this does not participate in the transmission of the processed image data 12. Rather, the CCM 4 only controls and regulates this transmission via the control connection 11 v). The entirety of the control connections 11, each designed as bidirectional data connections, each between the central control module CCM 4 and the respective connection partner, forms a local network 16, via which the CCM 4 can access the CCU 3, the peripheral devices 6a, 6b, and 6c, and the external server 23 in a controlling and regulating manner. In this manner, the CCM 4 can request, for example, parameters from the CCU 3 or one of the peripheral devices 6, in each case without a detour via another device, namely via the respective direct control connection 11.

On the basis of the reference sign 25, which symbolizes the separation between the local network 16 and external instances such as the server 23 or the Internet, it can also be comprehended that the CCM 4 adopts a gateway function: This is because the CCM 4 can provide, via the control connection 11 ii), for example, an access to the server 23 or to the Internet for one of the peripheral devices 6, for example, in order to carry out a software update of this respective system component.

The CCM 4 can also implement network-based functions, however. For example, in addition to the central control function, the CCM 4 can also exchange DICOM objects with the server 23 via the control line 11. Furthermore, the CCM 4 can also instruct the CCU 3 via the control connection 11 v) to transmit image data via the additional data connection 26 (dotted line in FIG. 2), which is not part of the network 16, to the server 23 (path i)).

Alternatively thereto, a local network can also be established with the aid of the CCM, via which local network the CCM can then communicate with all other devices located in the network (CCUs and peripheral devices). The CCM can additionally also establish a connection to a higher-order network (for example, of a hospital). In such an architecture, terminals, in particular the individual CCUs, do not have to deliver data to a PACS. Above-explained connection i) can then be saved accordingly. In this approach, there is thus only one connection into the higher-order (hospital) network/to the PACS, in order to implement a cost-effective architecture.

The CCM 4 also has the option on the basis of a touchscreen 15 to receive input commands from a user and convert them into corresponding actuation signals, for example, to thus actuate one of the peripheral devices 6. In this manner, the user can actuate and operate, for example, a light source or an OP instrument as the respective peripheral device 6 centrally via the CCM 4. The user can additionally have operating parameters and/or statuses of the respective peripheral device 6 displayed on the display device 9. In other words, the touchscreen 15 is used here as a “surgical cockpit”, using which the user can centrally control and regulate all relevant functions of the visualization system 1.

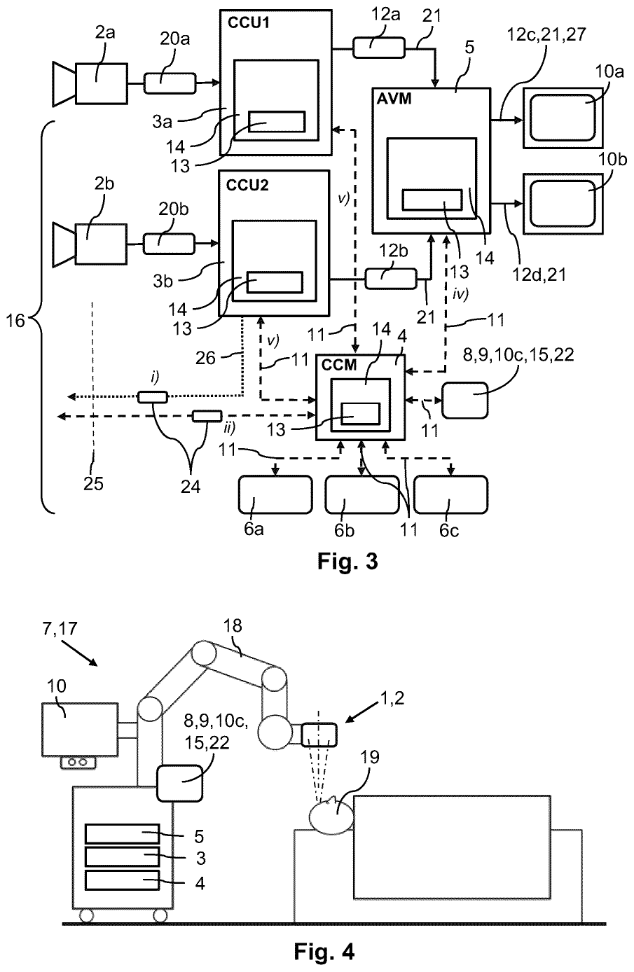

FIG. 3 shows a further visualization system 1 according to the invention, which essentially differs from the preceding example of FIG. 2 in that now two image recording devices 2a and 2b are provided here, which are each read out by an associated CCU 3a, 3b. There are also again respective control connections 11 v), via which the CCM 4 can control and bidirectionally communicate with the two CCUs 3a, 3b, in the example of FIG. 3. In contrast to the preceding example of FIG. 2, however, the processed image data 12a and 12b supplied by the two camera control units 3a, 3b are not output directly at a monitor here, but rather transferred in the form of respective video signals 21 to an advanced video processing module (AVM) 5.

The AVM 5 is also centrally controlled via the control connection 11 iv) by the CCM 4. For example, the user can specify via the touchscreen 15 in which form the AVM 5 is to process the respective video signals 21 in order to generate advanced image data 27 therefrom and output these data in the form of a live video image data stream 21 at the respective monitor 10a, 10b. Furthermore, mediated via the CCM 4, the AVM 5 can be instructed to arrange the received video image data streams 12a, 12b spatially adjacent to one another in a specific manner and possibly augment them with additional information in order to thus offer the user an augmented view of an operation scene.

FIG. 4 shows how a visualization system 1 designed according to the invention can be used during a surgical intervention, which is carried out using an operating system 7 comprising a surgery robot having a movable robot arm 18. It can be seen in FIG. 4 that the head of the patient 19 is visualized/observed with the aid of the visualization system 1, which can be moved in space on the movable robot arm 18, wherein a live video image of the operation area is displayable on the illustrated monitor 10 for the neurosurgeon. The neurosurgeon can transmit control commands via the touchscreen 15 to the CCM 4 in this case, which converts them into corresponding actuation signals and transmits these actuation signals via corresponding control connections 11 (as illustrated in FIG. 2 and FIG. 3) to the further components of the visualization system 3, 2, 6 and possibly 23.

Finally, it is also to be mentioned that the CCM 4 shown in FIG. 3 implements a method according to claim 10, since the CCM 4, in the context of the central control function, instructs the two CCUs 3a, 3b via the described control connection 11 v) to each transmit the processed image data 12 to the respective monitor 10a, 10b, wherein the transmission takes place mediated via the AVM 5, as described above with reference to FIG. 3.

In summary, a novel modular system architecture for a medical visualization system 1 is proposed, which provides a central control module 4, using which it is possible to access numerous components 3, 2, 6 and possibly 23 in a controlling manner, in particular a camera control unit 3 and possibly peripheral devices 6, in order to be able to exert a central control function in the sense of a “surgical cockpit” in this manner. With the aid of the CCM 4, the user, possibly with the aid of a touchscreen 15, can control all important functions of the visualization system 1 in a controlling manner, wherein the visualization system 1 can be modularly expanded using further components, which can then each be newly connected to the CCM 4 (cf. FIG. 3). This architecture is therefore based on a central control module (4), using which a central control function is executable. The visualization system 1 can be designed in this case in particular as a live video system.

### LIST OF REFERENCE SIGNS

1 visualization system
2 image recording device (in particular designed as a video camera)
3 camera control unit (CCU; for reading out and/or actuating 2)
4 central control module (CCM)
5 advanced video processing module (AVM)
6 peripheral device (anesthesia device, insufflator, pump, OP instrument, light source, etc.)
7 OP system (comprising 1 and at least one 6)
8 input unit (of 4)
9 display device (of 4)
10 monitor (for displaying a live video image data stream, which is recorded using 2)
11 control connection (enables control and, preferably bidirectional, communication between the connection partners, e.g., 4/3 or 4/5 or 4/6 or 4/23)
12 processed image data
13 software
14 processor
15 touchscreen
16 local network (for example, LAN)
17 surgery robot
18 movable robot arm
19 patient
20 (unprocessed) image data
21 video signal/video image data stream
22 user interface
23 server
24 database object (for example, DICOM object)
25 gateway (=interface to Internet/external or local network/database)
26 additional data connection (for example, between 3 and 23), not part of 16
27 advanced image data

1. A medical visualization system (1), comprising:
at least one camera control unit (3), which is configured to read image signals or image data (20) from at least one associated image recording device (2) and to generate respective processed image data (12) from the respective image signals/image data (20); and
a central control module (4), adapted to allow a user to access the at least one camera control unit (3) in a controlling manner in order to have the processed image data (12) displayed on at least one monitor (10), wherein
the central control module (4) being configured to provide a central control function with which, via a respective control connection (11), the at least one camera control unit (3) is adapted to be instructed to transmit the processed image data (12) to a monitor (10).

2. The visualization system (1) as claimed in claim 1, wherein the at least one camera control unit (3) is adapted to be instructed to transmit the processed image data (12) to the monitor (10) without the processed image data (12) arriving at the central control module (4).

3. The visualization system (1) as claimed in claim 1, wherein the central control module (4) is configured to access in a controlling and regulating manner, via a local network (16), the at least one camera control unit (3) and at least one peripheral device (6).

4. The visualization system (1) as claimed in claim 3, wherein the at least one peripheral device (6) includes at least one of a light source, an anesthesia device, an insufflator, a pump, or an OP instrument, and for this purpose multiple respective bidirectional control connections (11) are formed in the network (16) between the central control module (4) and further devices (2,3,5,6,7) of the visualization system (1).

5. The visualization system (1) as claimed in claim 4, wherein at least one of a) the central control module (4) is not configured to receive image data, and only to regulate in a controlling manner a flow of image data between individual components of the visualization system (1), or b) the central control module (4) is configured to request parameters from at least one of the at least one camera control unit (3) or from the at least one peripheral device (6) via respective direct control connections (11).

6. The visualization system (1) as claimed claim 1, wherein the central control module (4) is configured, as a gateway function, to provide at least one of: an internet access, an access to a local database, or an access to a cloud service, for at least one of the at least one camera control unit (3) or at least one peripheral device (6).

7. The visualization system (1) as claimed in claim 1, wherein the central control module (4) is configured to offer at least one of:
network-based functions such as DICOM (digital imaging and communications in medicine),
a central control function for the visualization system (1), or
at least one auxiliary function,
to a user of the visualization system (1).

8. The visualization system (1) as claimed in claim 7, wherein the central control module (4) is configured such that a user can operate at least one of the network-based functions, the central control function or the at least one auxiliary function via a user interface (22) of the central control module (4).

9. The visualization system (1) as claimed in claim 1, wherein the central control module (4) has at least one of a separate input unit (8) or separate display device (9) in order to enable the central control function, so that a user can access the at least one camera control unit (3) in a controlling manner via the input unit.

10. The visualization system (1) as claimed in claim 9, wherein at least one piece of external information, which is acquired from at least one of the camera control unit (3) or a peripheral device (6) connected to the control module (4), is displayable on the display device (9).

11. The visualization system (1) as claimed in claim 1, further comprising:
an advanced video processing module (AVM) 5, which is configured to receive already preprocessed image data from the at least one camera control unit (3) and to generate advanced image data therefrom by a downstream video processing function,
wherein the advanced video processing module (5) is a structural unit separate from the central control module (4).

12. The visualization system (1) as claimed in claim 11, wherein the central control module (4) is adapted to access the advanced video processing module (5) in a controlling manner via one of the control connections (11).

13. The visualization system (1) as claimed in claim 11, wherein the advanced video processing module (5) is configured to receive multiple video image data streams from assigned ones of the camera control units (3) of the visualization system (1) and to at least one of offset the received video image data streams (12a, 12b) with one another, scale the received video image data streams (12a, 12b), or specify a spatial arrangement of at least two of the received video image data streams.

14. The visualization system (1) as claimed in claim 13, wherein the advanced video processing module (5) is configured to at least one of a) superimpose multiple live video images, or b) augment at least one live video image data stream (21) with at least one of additional objects or an additional piece of information.

15. A medical operating system (7) for carrying out a medical intervention or a medical examination, the medical operating system (7) comprising
the medical visualization system (1) as claimed in claim 1; and
at least one peripheral device (6), which is at least one of readable, controllable, or operable mediated via the central control module (4);
in particular with the aid of an input unit (8) and/or a display device (9) of the central control module (4).

16. The medical operating system (7) of claim 15, wherein the at least one peripheral device (6) is at least one of readable, controllable, or operable via at least one of an input unit (8) or a display device (9) of the central control module (4).

17. A method for visualizing at least one video image data stream, which is recorded using at least one image recording device (2), the method comprising:
with the aid of at least one camera control unit (3), generating processed image data (12) from image signals or image data (20) of the at least one image recording device (2) as preprocessing;
displaying the processed image data (12) or advanced image data (27) generated therefrom on a monitor (10) as a video image data stream,
using a central control function of a central control module (4) via a respective control connection (11), instructing the at least one camera control unit (3) to transmit the processed image data (12) to the monitor (10).

18. The method as claimed in claim 17, further comprising generating the advanced image data (27) with an advanced video processing module (5) as downstream video processing, and transmitting, via the advanced video processing module (5), the advanced image data (27) to the monitor (10), wherein this transmitting and/or the downstream video processing is controlled via a control connection (11) using the central control module (4).

19. The method as claimed in claim 17, further comprising, for the visualization of the processed image data (12) or the advanced image data (27), via the central control module (4), at least one of:
accessing, in a controlling manner, the at least one camera control unit (3) and/or the advanced video processing module (5) based on a piece of external information of a peripheral device (6), which is displayed on a display device (9) of the central control module (4), or
accessing in a controlling manner a peripheral device (6) connected to the central control module (4) in order to thus influence imaging of the at least one image recording device (2).
